(19) United States
(12) Patent Application Publication
(10) Pub. No.: US 2005/0032741 A1
(43) Pub. Date: Feb. 10, 2005

(54) Vitamin Compositions

(76) Inventor: Balaji Venkataraman, Alpharetta, GA (US)

Correspondence Address: JOHN S. PRATT, ESQ;KILPATRICK STOCKTON, LLP, 1100 PEACHTREE STREET, ATLANTA, GA, 30309, US

(21) Appl. No.: 10/635,928

(22) Filed: Aug. 6, 2003

(51) Int. Cl. A61K 31/714; A61K 31/59; A61K 31/525; A61K 33/06

(52) U.S. Cl. 514/52; 514/167; 514/251; 424/682

(57) ABSTRACT

Provided are vitamin compositions and methods for the treatment or prevention of conditions associated with hormonal changes in an individual. The vitamin compositions contain calcium, vitamin D, folic acid, vitamin B12 and vitamin B6. In a preferred embodiment, the vitamin B12 is a hydroxocobalamin.

### FIELD OF THE INVENTION

The present invention relates generally to the fields of nutrition and the treatment or prevention of a condition in an individual associated with a hormonal change. Specifically, the invention relates to vitamin compositions comprising calcium, vitamin D, folic acid, vitamin B12 and vitamin B6.

### BACKGROUND OF THE INVENTION

Recent evidence has shown a very high prevalence of sub-optimal vitamin levels in diets (Flood et al., J. Natl. Cancer Inst. 2000; 92:1706). A recent article in JAMA concluded, “it appears to be prudent for all adults to take vitamin supplementation” (Fletcher et al., JAMA 2002, 287(23):3127). While poor dietary intake of vitamins remains the primary reason for this prevalence, many vitamins can be depleted due to other factors such as alterations in hormone levels, menopause, aging, alcohol consumption, smoking, genetics, environmental factors, and consumption of certain prescription medications. Certain vitamin deficiencies have also been linked with chronic diseases including coronary artery disease, osteoporosis, hyperhomocysteinemia and cancer. Many post menopausal women are affected by increased risks associated with hyperhomocysteinemia, osteoporosis, dementia, and cancer. Gonadal steroid treatment may affect some of these conditions.

The Women's Health Initiative (WHI), a double-blind, placebo-controlled trial studying the effects of continuous combined estrogen-progestin regimen (Conjugated Equine Estrogens (CEE) 0.625 mg plus 2.5 mg Medroxy Progesterone Acetate (MPA) daily) was stopped prematurely on the basis of an increase in the risk of invasive breast cancer, myocardial infarction and stroke (JAMA 2002: 288, 321). While many previous observational studies actually demonstrated cardiovascular benefits in women using other types or regimens of hormones, there seems to be a consensus on the interpretation of the WHI trial: 1) hormones are the best treatment for symptomatic women since there are no real alternatives; 2) women who use hormone replacement therapy (HRT), either estrogen therapy or estrogen-progesterone therpy, for more than 5 years should discuss the latest data with their physician, in order to consider their individual risk-benefit equation; and 3) it is logical to prefer hormones, which are different from CEE plus MPA daily. However, the practical implications of the WHI are that most physicians require patients to sign informed consents and continue medication at prior levels or at lower dosages and patients have opted out of taking estrogen (E) or estrogen-progesterone (P) combinations and are seeking natural alternatives. Since so many women are now seeking alternatives to traditional hormone therapy, there is a need for appropriate vitamin compositions for the treatment of menopausal women. These vitamin compositions should also treat other conditions most commonly affecting menopausal women such as hyperhomocysteinemia, osteoporosis and cancer.

Accordingly, there is a need for non-invasive vitamin-based treatments and preventatives for conditions commonly affecting menopausal women and all other individuals experiencing hormonal changes.

### SUMMARY OF THE INVENTION

The present invention addresses the problems described above by providing vitamin compositions and methods for the treatment or prevention of conditions associated with hormonal changes, including the treatment of menopausal females.

The vitamin compositions comprise calcium, vitamin D, folic acid, vitamin B12, vitamin B6, and in certain compositions additionally, vitamin C and or iron. The vitamin compositions may contain calcium, vitamin D, folic acid, vitamin B12 and vitamin B6 in any combination of amounts as follows: calcium in an amount less than 800 mg, preferably between about 200 mg and 800 mg, and more preferably about 400 mg; vitamin D in an amount less than 800 IU, preferably between about 300 IU and 500 IU, and more preferably about 400 IU; folic acid in an amount between about 0.8 mg and 5 mg and preferably about 1.6 mg; vitamin B12 in an amount between about 300 mcg and 2000 mcg, preferably between about 300 mcg and 1200 mcg, and more preferably about 500 mcg; and vitamin B6 in an amount between 10 mg and 100 mg and preferably about 25 mg.

In one embodiment of the present invention, the vitamin composition contains folic acid in an amount of about 1.6 mg, vitamin B12 (hydroxocobalamin) in an amount of about 500 mcg, vitamin B6 (pyridoxine) in an amount of about 25 mg, vitamin D3 in an amount of about 400 IU, and calcium (as carbonate) in an amount of about 400 mg. In other embodiments, the vitamin composition further comprises vitamin C in an amount less than 200 mg, preferably between about 30 mg and 100 mg, and more preferably about 60 mg; and or iron in an amount of between about 20 mg and 75 mg, and preferably 30 mg.

The present invention further includes methods of treating or preventing conditions associated with a hormonal change in an individual comprising administering to the individual an effective amount of a vitamin composition containing a calcium, vitamin D, folic acid, vitamin B12 and vitamin B6. In a preferred embodiment, the B12 vitamin is a cyanocobalamin. In another preferred embodiment, the B12 vitamin is a hydroxocobalamin. In another preferred embodiment, the B12 vitamin is a combination of cyanocobalamin and hydroxocobalamin.

Hormonal changes may occur in an individual due to treatment of the individual with estrogen, androgen or estrogen-androgen combination therapies or other long-term steroid treatments. Hormonal changes may also occur in an individual due to menopause, smoking, excercise, cancer chemotherapy, or ovariectomy/hysterectomy. Such hormonal changes can increase the individual's risk of hot flashes, high-risk pregnancy, bone loss, osteoporosis, cardiovascular disease, or osteopenia. In addition, patients suffering from endometriosis and uterine fibroids are at increased risk of similar problems. Accordingly, the vitamin compositions of the present invention are administered to individuals including those who have undergone or are undergoing treatment with cancer chemotherapy, estrogen, androgen, estrogen-androgen combination therapies, progesterone, estrogen-progesterone combination therapies or other steroids; are or were smokers; or are experiencing or have experienced menopause, high risk pregnancy, ovariectomy/hysterectomy, endometriosis, bone loss, osteoporosis, uterine fibroids, cardiovascular disease or osteopenia.

It is an object of the present invention to provide a vitamin composition comprising calcium, vitamin D, folic acid, vitamin B12 and vitamin B6.

It is another object of the present invention to provide a vitamin composition comprising calcium, vitamin D, folic acid, vitamin B12, vitamin B6 and vitamin C.

It is still another object of the present invention to provide a vitamin composition comprising calcium, vitamin D, folic acid, vitamin B12, vitamin B6 and iron.

It is a further object of the present invention to provide a vitamin composition containing vitamin B12 in a hydroxocobalamin form.

It is another object of the present invention to provide a vitamin composition containing vitamin B12 in a cyanocobalamin form.

It is a further object of the present invention to provide a vitamin composition containing vitamin B12 in a combination of both a hydroxocobalamin form and a cyanocobalamin form.

It is a still further object of the present invention to provide a vitamin composition for the treatment or prevention of a condition associated with a hormonal change in an individual, wherein the vitamin composition comprises calcium, vitamin D, folic acid, vitamin B12 and vitamin B6.

It is yet another object of the present invention to provide methods for treating or preventing a condition associated with a hormonal change in an individual.

It is another object of the present invention to provide methods for treating or preventing a condition associated with a hormonal change in an individual comprising administering to the individual the vitamin composition of the present invention.

It is yet another object of the present invention to provide methods for administering the vitamin compositions of the present invention to individuals who have undergone or are undergoing treatment with estrogen, estrogen-progesterone therapies, androgen, estrogen-androgen combination therapies, or steroids; are or were smokers; or are experiencing or have experienced menopause, high risk pregnancy, endometriosis, osteoporosis or uterine fibroids.

These and other objects, features and advantages of the present invention will become apparent after a review of the following detailed description of the disclosed embodiments and claims.

### DETAILED DESCRIPTION OF THE INVENTION

The present invention provides new and effective vitamin compositions and methods for the treatment or prevention of conditions associated with a hormonal change in an individual, including the treatment of menopausal females.

Vitamin Compositions

The vitamin compositions of the present invention comprise calcium, vitamin D, folic acid, vitamin B12 and vitamin B6. In other embodiments, the vitamin compositions also comprise vitamin C and or iron. As used herein, the term “vitamin B12” refers to all forms of cobalamin including, but not limited to, hydroxocobalamin, cyanocobalamin and methylcobalamin. Accordingly, the vitamin compositions of the present invention can include one or more forms of vitamin B12 including hydroxocobalamin, cyanocobalamin and methylcobalamin. In a preferred embodiment, the vitamin compositions include one form of vitamin B12, which form is hydroxocobalamin. In another preferred embodiment, the vitamin compositions include one form of vitamin B12, which form is cyanocobalamin. As also used herein, the term “vitamin B6” refers to pyridoxal, pyridoxamine and pyridoxine compounds. The term “vitamin D” is used to refer to both cholecalciferol (vitamin D3) and ergocalciferol (vitamin D2). In a preferred embodiment, the vitamin D is a including calcium carbonate, phosphate, lactate, gluconate, citrate and combinations thereof. The term “vitamin C” is used herein to refer to any form of vitamin C including ascorbate and L threonate. The term “iron” is used to refer to any form of iron including gluconate, sulfate, chloride, elemental and fumarate. It is also to be understood that as used in the specification and in the claims, “a” or “an” can mean one or more, depending upon the context in which it is used.

The vitamin compositions described herein can contain calcium in an amount less than 600 mg, preferably between about 200 mg and 600 mg, and more preferably about 400 mg. In another or further embodiment, the vitamin composition contains vitamin D in an amount less than 600 IU, preferably between about 300 IU and 500 IU, and more preferably about 400 IU. A vitamin D3 is preferred. In another or further embodiment, the vitamin composition contains folic acid in an amount between about 0.8 mg and 5 mg and preferably about 1.6 mg. In another or further embodiment, the vitamin composition contains vitamin B12 in an amount between about 300 mcg and 2000 mcg, preferably between about 300 mcg and 1200 mcg, and more preferably about 500 mcg. The vitamin B12 may be hydroxy-, cyano- or methylcobalamin, but is preferably hydroxocobalamin. In another or further embodiment, the vitamin composition contains vitamin B6 in an amount between about 10 mg and 100 mg and preferably about 25 mg. In another or further embodiment, the vitamin composition contains vitamin C in an amount less than 200 mg, preferably between about 30 mg and 100 mg, and more preferably about 60 mg. In another or further embodiment, the vitamin composition contains iron in amount of between about 20 mg to 75 mg, and preferably about 30 mg.

Accordingly, the vitamin compositions comprise calcium, vitamin D, folic acid, vitamin B12 and vitamin B6 in any combination of amounts as follows: calcium in an amount less than 800 mg, preferably between about 200 mg and 800 mg, and more preferably about 400 mg; vitamin D in an amount less than 800 IU, preferably between about 300 IU and 500 IU, and more preferably about 400 IU; folic acid in an amount between about 0.8 mg and 5 mg and preferably about 1.6 mg; vitamin B12 in an amount between about 300 mcg and 2000 mcg, preferably between about 300 mcg and 1200 mcg, and more preferably about 500 mcg; and vitamin B6 in an amount between 10 mg and 100 mg, and preferably about 25 mg. The vitamin compositions may further comprise any combination of the calcium, vitamin D, folic acid, vitamin B12, and vitamin B6 as provided above in addition to any combination of amounts of vitamin C and or iron as follows: vitamin C in an amount less than 200 mg, preferably between about 30 mg and 100 mg, and more preferably about 60 mg; and iron in an amount of between about 20 mg to 75 mg, and preferably about 30 mg.

In one embodiment, vitamin compositions of the present invention comprise one of calcium, vitamin D, folic acid, vitamin B12 or vitamin B6 in an amount prescribed above and the other four listed vitamins in any amount. For example, one vitamin composition of the present invention contains calcium in an amount less than 800 mg, preferably between about 200 mg and 800 mg, and more preferably about 400 mg along with each of vitamin D, folic acid, vitamin B12 and vitamin B6 in any amount. Another vitamin composition contains vitamin D in an amount less than 800 IU, preferably between about 300 IU and 500 IU, and more preferably about 400 IU along with each of calcium, folic acid, vitamin B12 and vitamin B6 in any amount. Another vitamin composition contains folic acid in an amount between about 0.8 mg and 5 mg and preferably about 1.6 mg along with each of calcium, vitamin D, vitamin B12 and vitamin B6 in any amount. Another vitamin composition contains vitamin B12 in an amount between about 300 mcg and 2000 mcg, preferably between about 300 mcg and 1200 mcg, and more preferably about 500 mcg along with each of calcium, vitamin D, folic acid and vitamin B6 in any amount. Another vitamin composition contains vitamin B6 in an amount between about 10 mg and 100 mg and preferably about 25 mg along with each of calcium, vitamin D, folic acid and vitamin B12 in any amount.

Other vitamin compositions additionally contain vitamin C and or iron. Accordingly, another vitamin composition contains vitamin C in an amount less than 200 mg, preferably between about 30 mg and 100 mg, and more preferably about 60 mg along with each of calcium, vitamin D, folic acid, vitamin B12 and vitamin B6 in any amount. Still another another vitamin composition contains vitamin C in an amount less than 200 mg, preferably between about 30 mg and 100 mg, and more preferably about 60 mg along with each of calcium, vitamin D, folic acid, vitamin B12, vitamin B6 and iron in any amount. Another vitamin composition contains iron in an amount of between about 20 mg and 75 mg, and preferably about 30 mg, along with each of calcium, vitamin D, folic acid, vitamin B12, and vitamin B6 in any amount. Still another vitamin composition contains iron in an amount of between about 20 mg and 75 mg, and preferably 30 mg, along with each of calcium, vitamin D, folic acid, vitamin B12, vitamin B6 and vitamin C in any amount.

Other vitamin compositions of the present invention comprise two of calcium, vitamin D, folic acid, vitamin B12 and vitamin B6 in an amount prescribed above and the other three listed vitamins in any amount. Still other vitamin compositions of the present invention comprise three of calcium, vitamin D, folic acid, vitamin B12 and vitamin B6 in an amount prescribed above and the other two listed vitamins in any amount. Further vitamin compositions comprise four of calcium, vitamin D, folic acid, vitamin B12 and vitamin B6 in an amount prescribed above and the other one listed vitamin in any amount. In some embodiments, vitamin compositions comprise all five of calcium, vitamin D, folic acid, vitamin B12 and vitamin B6 in an amount prescribed above. In other embodiments, vitamin compositions comprise all five of calcium, vitamin D, folic acid, vitamin B12 and vitamin B6 in an amount prescribed above and vitamin C and or iron in any amount or in an amount prescribed above.

In one embodiment of the present invention, the vitamin composition contains folic acid in an amount of about 1.6 mg, hydroxocobalamin in an amount of about 500 mcg, pyridoxine in an amount of about 25 mg, calcium (as carbonate) in an amount of about 400 mg, vitamin D3 in an amount of about 400 IU. In another embodiment, the vitamin composition contains folic acid in an amount of about 1.6 mg, hydroxocobalamin in an amount of about 500 mcg, pyridoxine in an amount of about 25 mg, calcium (as carbonate) in an amount of about 400 mg, vitamin D3 in an amount of about 400 IU, vitamin C in an amount of about 60 mg, and iron in an amount of between about 30 mg.

In another embodiment of the present invention, the vitamin composition contains folic acid in an amount of about 1.6 mg, vitamin B12 (cyanocobalamin in an amount from 1 to 499 mcg and hydroxocobalamin in an amount from 1 to 499 mcg provided that the total amount of cyanocobalamin and hydroxocobalamin does not exceed 500 mcg), vitamin B6 (pyridoxine) in an amount of about 25 mg, vitamin D3 in an amount of about 400 IU, and calcium (as carbonate) in an amount of about 400 mg. In yet another embodiment of the present invention, the vitamin composition contains folic acid in an amount of about 1.6 mg, vitamin B12 (cyanocobalamin in an amount of about 250 mcg and hydroxocobalamin in an amount of about 250 mcg), vitamin B6 (pyridoxine) in an amount of about 25 mg, vitamin D3 in an amount of about 400 IU, and calcium (as carbonate) in an amount of about 400 mg. In some embodiments, the vitamin compositions further comprise vitamin C in an amount of about 60 mg, and iron in an amount of between about 30 mg.

Auxiliary Agents

The vitamin compositions of the present invention may be a part of a vitamin formulation that also contains at least one of any suitable auxiliary such as, but not limited to, diluent, binder, stabilizer, buffers, salts, lipophilic solvents, preservative or the like. Pharmaceutically acceptable auxiliaries are preferred. Examples and methods of preparing such sterile solutions are well known in the art and can be found in well-known texts such as, but not limited to, REMINGTON'S PHARMACEUTICAL SCIENCES (Gennaro, Ed., 18th Edition, Mack Publishing Co. (1990)). Pharmaceutically acceptable carriers can be routinely selected that are suitable for the mode of administration, solubility and/or stability of the compound.

Pharmaceutical excipients and additives useful in the present invention include, but are not limited to, proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination in ranges of 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like.

Carbohydrate excipients suitable for use in the present invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, sorbitol (glucitol), myoinositol and the like.

The vitamin compositions of the present invention can also be a part of a vitamin formulation that includes a buffer or a pH-adjusting agent. Typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers.

Additionally, vitamin compositions of the invention can be a part of a vitamin formulation that includes polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, anti-microbial agents, sweeteners, antioxidants, anti-static agents, surfactants (e.g., polysorbates such as “TWEEN 20” and “TWEEN 80”), lipids (e.g., phospholipids, fatty acids), steroids (e.g., cholesterol), and chelating agents (e.g., EDTA). These and additional known pharmaceutical excipients and/or additives suitable for use in the present invention are known in the art, e.g., as listed in REMINGTON: THE SCIENCE & PRACTICE OF PHARMACY (19^{th} ed., Williams & Williams (1995)) and PHYSICIAN'S DESK REFERENCE (52^{nd} ed., Medical Economics (1998)), the disclosures of which are expressly entirely incorporated herein by reference.

Vitamin Formulations for Oral Administration

For oral administration in the form of a tablet or capsule, the vitamin compositions described herein (any combination of specified amounts or ranges of calcium, vitamin D, folic acid, vitamin B12, vitamin B6 and optionally vitamin C ad or iron) may be combined with an oral, non-toxic pharmaceutically acceptable inert carrier such as ethanol, glycerol, water and the like to create a vitamin formulation. Moreover, when desired or necessary, suitable binders, lubricants, disintegrating agents, flavoring and coloring agents may also be incorporated into the mixture. Suitable binders include, without limitation, starch; gelatin; natural sugars such as glucose or beta-lactose; corn sweeteners; natural and synthetic gums such as acacia, tragacanth, or sodium alginate, carboxymethylcellulose; polyethylene glycol; waxes and the like. Lubricants used in these dosage forms include, without limitation, sodium oleate, sodium stearate, calcium stearate, sodium benzoate, sodium acetate, sodium chloride and the like. Disintegrators include, without limitation, starch, methyl cellulose, agar, bentonite, xanthan gum and the like.

In one embodiment, a vitamin formulation suitable for oral administration contains carnauba wax, citric acid, dicalcium phosphate, hydroxypropyl methylcellulose, calcium stearate, microcrystalline cellulose, polyethylene glycol, polysorbate 80, riboflavin, silicon dioxide, sodium benzoate, sodium citrate, sodium starch glycolate, sorbic acid, starch, stearic acid and titanium dioxide.

Vitamin compositions or formulations of the present invention suitable for oral administration may be presented as discrete units such as capsules, cachets or tablets each containing a predetermined amount of a vitamin ingredient; as a powder or granules; as a solution or a suspension in an aqueous liquid or a non-aqueous liquid; or as an oil-in-water liquid emulsion or a water-in-oil emulsion and as a bolus, etc. In a preferred embodiment, the vitamin composition or formulation is in the form of a tablet.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated so as to provide a slow or controlled release of the vitamin ingredient(s) therein.

Preferred unit dosage formulations are those containing a daily dose or unit, daily sub-dose, or an appropriate fraction thereof, of the administered ingredient. In a more preferred embodiment, the unit dosage formulation contains from one daily dose or half of a daily dose of the vitamin compositions described above.

Vitamin Formulations for Other Routes of Administration

Formulations suitable for parenteral administration include aqueous and non-aqueous sterile injection solutions which may contain anti-oxidants, buffers, bacteriostats and solutes that render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

For parenteral administration, sterile suspensions and solutions are desired. Isotonic preparations, which generally contain suitable preservatives, are employed when intravenous administration is desired. The vitamin compositions may be administered parenterally via injection of a formulation consisting of the active vitamin ingredients dissolved in an inert liquid carrier. The term “parenteral,” as used herein, includes, but is not limited to, subcutaneous injections, intravenous, intramuscular, intraperitoneal injections, or infusion techniques. Acceptable liquid carriers include, for example, vegetable oils such as peanut oil, cottonseed oil, sesame oil and the like, as well as organic solvents such as solketal, glycerol formal and the like. Dissolving or suspending the vitamin composition in the liquid carrier such that the final formulation contains from about 0.005% to 30% by weight of the active ingredient, i.e., a vitamin composition of the present invention.

Formulations suitable for topical administration in the mouth include lozenges comprising the ingredients in a flavored basis, usually sucrose and acacia or tragacanth; pastilles comprising the vitamin composition in an inert basis such as gelatin and glycerin, or sucrose and acacia; and mouthwashes comprising the vitamin composition to be administered in a suitable liquid carrier. The liquid forms may include suitably flavored suspending or dispersing agents such as the synthetic and natural gums, for example, tragacanth, acacia, methyl-cellulose and the like.

Formulations suitable for nasal administration, wherein the carrier is a solid, include a coarse powder having a particle size, for example, in the range of 20 to 500 microns which is administered in the manner in which snuff is taken, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the vitamin composition.

The vitamin compositions may also be entrapped in microcapsules prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions. REMINGTON'S PHARMACEUTICAL SCIENCES (A. Osol ed., 16th ed. (1980)).

In addition, the vitamin compositions may be incorporated into biodegradable polymers allowing for sustained release of the compound, the polymers being implanted in the vicinity of where vitamin delivery is desired, for example, within the buccal cavity. The biodegradable polymers and their uses are described, for example, in detail in Brem et al., 74 J. NEUROSURG. 441-46 (1991). Suitable examples of sustained-release compositions include semipermeable matrices of solid hydrophobic polymers containing a vitamin composition of the present invention, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT® (Tap Pharmaceuticals, Inc., Chicago, Ill.) (injectable microspheres composed of lactic acid glycolic acid copolymer and leuprolide acetate), and poly-D-(−)-3-hydroxybutyric acid.

Pharmaceutically Acceptable Preservatives

The present invention provides stable vitamin compositions as well as preserved vitamin solutions and formulations containing a preservative as well as multi-use preserved formulations suitable for pharmaceutical or veterinary use, comprising a vitamin composition disclosed herein in a pharmaceutically acceptable formulation. Formulations in accordance with the present invention may optionally contain at least one known preservative. Preservatives include, but are not limited to, phenol, m-cresol, p-cresol, o-cresol, chlorocresol, benzyl alcohol, phenylmercuric nitrite, phenoxyethanol, formaldehyde, chlorobutanol, calcium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein. Non-limiting examples include, no preservative, 0.1-2% m-cresol, 0.1-3% benzyl alcohol, 0.001-0.5% thimerosal, 0.001-2.0% pheno, 0.0005-1.0% alkylparaben(s), and the like.

Other excipients, e.g., isotonicity agents, buffers, antioxidants, preservative enhancers, can be optionally added to the diluent. An isotonicity agent such as glycerin is commonly used at known concentrations. A physiologically tolerated buffer is preferably added to provide improved pH control. The formulations can cover a wide range of pHs, such as from about pH 4.0 to about pH 10.0, specifically, a range from about pH 5.0 to about pH 9.0, and more specifically, a range of about 6.0 to about 8.0. Suitable buffers include phosphate buffers, for example, sodium phosphate and phosphate buffered saline (PBS).

Methods of Treating or Preventing Conditions Associated with Hormonal Changes

The present invention further includes methods of treating or preventing a condition associated with a hormonal change in an individual comprising administering to the individual an effective amount of a vitamin composition comprising a calcium, vitamin D, folic acid, vitamin B12 and vitamin B6. In some embodiments, the vitamin composition further comprises vitamin C and or iron. An “individual” includes both a human and an animal. As used herein, the term “hormonal change” refers to any increase or decrease in a hormone within an individual and the term “condition” refers to any disease, illness, infection, or potentially detrimental change in the health of an individual. A condition “associated” with a hormonal change can be directly or indirectly caused by the hormonal change. Conditions associated with hormonal changes include, but are not limited to, conditions associated with menopause, hormone replacement therapy, ovariectomy/hysterectomy, cancer therapy, hot flashes, bone loss, high-risk pregnancy, osteoporosis, endometriosis, and uterine fibroids. “Treatment of” or “treating” a condition does not require elimination of the condition, i.e., curing of a disease.

Accordingly, an “effective amount” of a vitamin composition is defined herein as an amount of the vitamin composition capable of preventing or reducing the severity or occurrence of one or more conditions in an individual. In some embodiments, a vitamin composition described herein is administered to an individual in an amount effective to reduce the occurrence or severity of hot flashes, bone loss, high-risk pregnancy, osteoporosis, endometriosis, or uterine fibroids. In other embodiments, a vitamin composition described herein is administered to an individual in an amount effective to prevent hot flashes, bone loss, high-risk pregnancy, osteoporosis, endometriosis, or uterine fibroids. The individual described herein may be any individual, and in some embodiments is a pre-menopausal, menopausal or post-menopausal female.

A vitamin composition may also be administered to an individual in an amount effective to treat or prevent one or more of the following conditions: hyperhomocystineamia, “in-situ” vascular free radical formation and hypertension. In a preferred embodiment of the present invention, the vitamin compositions of the present invention treat or prevent hyperhomocystineamia, bone loss, “in-situ” vascular free radical formation and hypertension. Bone loss may or may not amount to osteoporosis as clinically defined.

Hormonal changes may occur in an individual due to treatment of the individual with estrogen, androgen or estrogen-androgen combination therapies or other long-term steroid treatments. One non-limiting example of an estrogen-androgen combination is Estratest, an estrogen-testosterone combination for improving sexual function. Hormonal changes may also occur in an individual due to menopause, smoking, excercise, cancer chemotherapy or ovariectomy/hysterectomy. Such hormonal changes can increase the individual's risk of hot flashes, high-risk pregnancy, bone loss, osteoporosis, cardiovascular disease, and osteopenia. In addition, patients suffering from endometriosis or uterine fibroids are at increased risk of similar problems. Accordingly, the vitamin compositions of the present invention are administered to individuals including those who have undergone or are undergoing treatment with cancer chemotherapy, estrogen, androgen, estrogen-androgen combination therapies, progesterone, estrogen-progesterone combination therapies or other steroids; are or were smokers; or are experiencing or have experienced menopause, high risk pregnancy, ovariectomy/hysterectomy, endometriosis, bone loss, osteoporosis, uterine fibroids, cardiovascular disease or osteopenia.

The vitamin compositions administered to these individuals and others comprise calcium, vitamin D, folic acid, vitamin B12 and vitamin B6 in any combination of amounts as follows: calcium in an amount less than 800 mg, preferably between about 200 mg and 800 mg, and more preferably about 400 mg; vitamin D in an amount less than 800 IU preferably between about 300 IU and 500 IU, and more preferably about 400 IU; folic acid in an amount between about 0.8 mg and 5 mg and preferably about 1.6 mg; vitamin B12 in an amount between about 300 mcg and 2000 mcg, preferably between about 300 mcg and 1200 mcg, and more preferably about 500 mcg; and vitamin B6 in an amount between 10 mg and 100 mg and preferably about 25 mg. The vitamin compositions administered to these individuals and others may further comprise any combination of the calcium, vitamin D, folic acid, vitamin B12, and vitamin B6 as provided above in addition to any combination of amounts of vitamin C and or iron as follows: vitamin C in an amount less than 200 mg, preferably between about 30 mg and 100 mg, and more preferably about 60 mg; and iron in an amount of between about 20 mg to 75 mg, and preferably 30 mg.

Routes of Administration

When treating a condition associated with a hormonal change in an individual, the vitamin compositions disclosed herein may be administered to the individual by any of the following routes: oral, parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, and transdermal means. In a preferred embodiment, a vitamin composition is administered orally.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### EXAMPLE 1

Preparation of Vitamin Composition

A vitamin composition is prepared that comprises folic acid USP 1.6 mg, vitamin B12 (hydroxocobalamin) USP 500 mcg, vitamin B6 (pyridoxine) USP 25 mg, calcium USP (as carbonate) 400 mg, and vitamin D3 USP 400 IU. A vitamin formulation containing the vitamin composition further contains camauba wax, citric acid, dicalcium phosphate, hydroxypropyl methylcellulose, magnesium stearate, microcrystalline cellulose, polyethylene glycol, polysorbate 80, riboflavin, silicon dioxide, sodium benzoate, sodium citrate, sodium starch glycolate, sorbic acid, starch, stearic acid and titanium dioxide.

### EXAMPLE 2

Administration of Vitamin Composition

The vitamin composition described in Example 1 is indicated for the distinctive nutritional requirements of: individuals with or at risk for cardiovascular disease, cerebrovascular disease, or osteoporosis, with particular emphasis on menopausal patients; patients treated with estrogen, androgen, estrogen-androgen combination therapies, or estrogen-progesterone combination therapies; smokers; patients with endometriosis or uterine fibroids; patients undergoing long term steroid treatments or cancer chemotherapy; and patients who have had a high-risk pregnancy, hysterectomy and or ovariectomy. Dosage and administration in adults is one to two tablets daily or as directed by a physician.

Accordingly, the vitamin composition described in Example 1 is orally administered to the patients described below in an amount of one to two tablets daily for various periods. The patients are monitored for osteoporsosis (Bone Mineral Desnsity Test), hyper homocysteinuria, and plaque formation (ECHO Cardiogram, computerized assisted tomography (CAT) scan or angiogram).

Patient 1

The patient is a 55-year-old female who is post menopausal for 3 months. The patient suffers from hot flashes. A fasting lipid profile obtained at a second visit showed high density lipoprotein (HDL) at 38 mg/dL and total cholesterol (TC) at 201 mg/dL. The patient is normotesive and the bone mineral index (BMI) shows propensity for osteoporosis. The patient is currently on low dose estrogen replacement therapy (ERT). Administration of the vitamin composition described in Example 1 improves the patient's blood pressure and decreases the rate of bone loss.

Patient 2

The patient is a 60-year-old female. The patient has recently chosen not to continue ERT and has since suffered briefly from vaginal dryness and has recently had memory lapses. The patient is mildly hypertensive (diastolic pressure (DP) 93) and has high low density lipoprotein (LDL 190). The patient has a familial history for breast cancer, but a mammogram is negative. The patient is currently taking phytoestrogens and low dose angiotensin receptor blocker (ARB). Administration of the vitamin composition described in Example 1 stabilizes memory loss.

Patient 3

The patient is a 36-year-old female who suffers from endometriosis. The patient recently had a male child. The patient suffers from excessive bleeding for the past six years prior to diagnosis of endometriosis. The patient is normotensive and is currently on gonadotropin releasing hormone (GnRH) antagonist depot. The patient is also severely anemic and recently had two fractures. BMI measurements reveal osteoporosis. The patient is currently taking Leuprolide depot and Fosomax. Administration of the vitamin composition described in Example 1 decreases the rate of bone loss and decreases the symptoms associated with endometriosis including excessive bleeding.

Patient 4

The patient is a 40-year-old female with a hysterectomy and removal of the ovaries. The patient smokes 25 to 30 cigarettes per day. The patient suffers from symptoms associated with removal of ovarian reproductive hormones and is concerned about osteoporosis. Radiologic examination of the cervical spine reveals loss of bone mineralization in the cervical vertebrae. The patient's physician begins administration of estrogen-progesterone combination therapy and daily administration of the vitamin composition described in Example 1. Administration of the vitamin composition described in Example 1 decreases the rate of bone loss in the cervical vertebrae and decreases the symptoms associated with removal of ovarian reproductive hormones.

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims.

1. A composition for treating a condition associated with a hormonal change comprising calcium, vitamin D, folic acid in an amount between about 0.8 mg and 5 mg, vitamin B12 and vitamin B6.

2. The composition of claim 1, wherein the calcium is in an amount less than 800 mg.

3. The composition of claim 1, wherein the calcium is in an amount between about 200 mg and 800 mg.

4. The composition of claim 1, wherein the vitamin D is a vitamin D3 and in an amount less than 800 IU.

5. The composition of claim 1, wherein the vitamin D is a vitamin D3 and in an amount between about 300 IU and 500 IU.

6. Canceled.

7. The composition of claim 1, wherein the vitamin B12 is a hydroxocobalamin.

8. The composition of claim 7, wherein the hydroxocobalamin is in an amount of between about 300 mcg and 2000 mcg.

9. The composition of claim 7, wherein the hydroxocobalamin is in an amount of between about 300 mcg and 1200 mcg.

10. The composition of claim 1, wherein the vitamin B6 is in an amount of between about 10 mg and 100 mg.

11. The composition of claim 1, wherein the calcium is in an amount between about 200 mg and 800 mg, vitamin D is in an amount between about 300 IU and 500 IU, vitamin B12 is in an amount of between about 300 mcg and 1200 mcg, and vitamin B6 is in an amount of between about 10 mg and 100 mg.

12. The composition of claim 1, wherein the calcium is in an amount of about 400 mg, vitamin D is in an amount of about 400 IU, folic acid is in an amount of about 1.6 mg, vitamin B12 is hydroxocobalamin in an amount of about 500 mcg, and vitamin B6 is in an amount of about 25 mg.

13. The composition of claim 1 further comprising vitamin C in an amount of less than about 200 mg.

14. The composition of claim 1 further comprising iron in an amount of between about 20 mg and 75 mg.

15. A method of treating a condition associated with a hormonal change in an individual comprising administering to the individual an effective amount of a vitamin composition comprising calcium, vitamin D, folic acid in an amount between about 0.8 mg and 5 mg, vitamin B12 and vitamin B6.

16. The method of claim 15, wherein the vitamin B12 is a hydroxocobalamin.

17. The method of claim 15, wherein the vitamin D is a vitamin D3.

18. The method of claim 15, wherein the calcium is in an amount between about 200 mg and 800 mg, vitamin D is in an amount between about 300 IU and 500 IU, vitamin B12 is in an amount of between about 300 mcg and 1200 mcg, and vitamin B6 is in an amount of between about 10 mg and 100 mg.

19. The method of claim 15, wherein calcium is in an amount of about 400 mg, vitamin D is in an amount of about 400 IU, folic acid is in an amount of about 1.6 mg, vitamin B12 is a hydroxocobalamin in an amount of about 500 mcg, and vitamin B6 is in an amount of about 25 mg.

20. The method of claim 15, wherein the composition further comprises vitamin C in an amount of less than 200 mg.

21. The method of claim 15, wherein the composition further comprises iron in an amount of between about 20 mg and 75 mg.

22. The method of claim 15, wherein the hormonal change is caused by menopause, smoking, hysterectomy, ovariectomy or cancer chemotherapy, or treatment of the individual with an estrogen, an androgen, an estrogen-androgen combination, an estrogen-progesterone combination, a steroid or a drug that affects the reproductive system.

23. A composition for treating a condition associated with a hormonal change comprising calcium, vitamin D, folic acid in an amount between about 0.8 mg and 5 mg, hydroxocobalamin and vitamin B6.

24. A method of treating a condition associated with a hormonal change in an individual comprising administering to the individual an effective amount of a vitamin composition comprising calcium, vitamin D, folic acid in an amount between about 0.8 mg and 5 mg, hydroxocobalamin and vitamin B6.

25. A composition for treating a condition associated with a hormonal change comprising calcium, vitamin D, folic acid, hydroxocobalamin in an amount between about 300 mcg and 2000 mcg, and vitamin B6.

26. The composition of claim 25, wherein the hydroxocobalamin is an amount of about 500 mcg.

27. A composition for treating a condition associated with a hormonal change comprising calcium, vitamin D, folic acid in an amount between about 0.8 mg and 5 mg, hydroxocobalamin in an amount between about 300 mcg and 2000 mcg, and vitamin B6.

28. The composition of claim 27, wherein the hydroxocobalamin is an amount between about 300 mcg and 1200 mcg.

29. The composition of claim 27, wherein the hydroxocobalamin is an amount of about 500 mcg.

30. A method of treating a condition associated with a hormonal change in an individual comprising administering to the individual an effective amount of a vitamin composition comprising calcium, vitamin D, folic acid, hydroxocobalamin in an amount of about 300 mcg and 1200 mcg, and vitamin B6.

31. The composition of claim 7, wherein the hydroxocobalamin is in an amount of about 500 mcg.

32. The method of claim 24 wherein the hydroxocobalamin is in an amount of about 500 mcg.

33. The method of claim 30 wherein the hydroxocobalamin is in an amount of about 500 mcg.
